(19) United States
(12) Patent Application Publication
(10) Pub. No.: US 2015/0176053 A1
(43) Pub. Date: Jun. 25, 2015

(54) TEST STRIP INSERTION DRIVE MECHANISM FOR ANALYTE METER

(71) Applicant: Cilag GmbH International, Zug (CH)

(72) Inventors: David Elder, Inverness (GB); Steven Setford, Inverness (GB); Allan Faulkner, Inverness (GB); Ryan Walsh, Douglassville, PA (US)

(73) Assignee: CILAG GMBH INTERNATIONAL, Zug (CH)

(21) Appl. No.: 14/138,798

(22) Filed: Dec. 23, 2013

(51) Int. Cl. C12Q 1/54

(52) U.S. Cl. CPC C12Q 1/54

(57) ABSTRACT

An analyte meter having a test strip port includes a detector proximate the port for detecting a test strip being inserted therein. A drive mechanism connected to the detector is configured to engage and pull a detected test strip into the test meter and into electrical and mechanical engagement therewith to enable analyte tests to be conducted.

### TECHNICAL FIELD

This application generally relates to the field of blood glucose measurement systems and more specifically to a test meter comprising a drive mechanism for enabling a test strip to be automatically inserted into a strip port connector for mechanical and electrical connection with the test meter.

### BACKGROUND

Systems that measure analytes in biological fluids, as exemplified by the determination of glucose in blood, typically comprise an analyte meter that is configured to receive a biosensor, usually in the form of a test strip. Because many of these systems are portable, and testing can be completed in a short amount of time, patients are able to use such devices in the normal course of their daily lives without significant interruption to their personal routines. A person with diabetes may measure their blood glucose levels several times a day as a part of a self management process to ensure glycemic control of their blood glucose within a target range.

There currently exist a number of available portable electronic devices that can measure glucose levels in an individual based on a small sample of blood. To perform an assay of the sample, a person is required to prick their finger and provide a blood sample on the test strip. The test strip is then inserted into a test strip port of the test meter to initiate an assay of the sample. Test strips oftentimes may be difficult to manipulate by users due to the small size of the test strips and limitations in the manual dexterity and visual impairment of some users. The user needs to properly align the test strip with the strip port connector and push the test strip in the correct direction to have a proper insertion, which, as mentioned above, can sometimes be problematic for users with dexterity problems. It would therefore be advantageous to provide a test meter that automatically inserts the test strip into its strip port connector.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention (wherein like numerals represent like elements).

FIG. 1A illustrates a diagram of an exemplary test strip based blood analyte measurement system, including a test meter and an analytical test strip;

FIG. 1B illustrates a diagram of an exemplary processing system of the test meter of FIG. 1A;

FIG. 2 illustrates a top perspective view of a portion of the test meter of FIG. 1A;

FIGS. 3A-3C illustrate sectioned views taken in sequence illustrating an exemplary drive mechanism of the test meter of FIGS. 1A and 2 in operation relative to a test strip; and

FIG. 4 illustrates a flow chart of exemplary steps performed by the test meter of FIG. 1A and more specifically the drive mechanism of FIGS. 3A-3C.

### MODES OF CARRYING OUT THE INVENTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “patient” or “user” refer to any human or animal subject and are not intended to limit the systems or methods to human use, although use of the subject invention in a human patient represents a preferred embodiment.

The term “sample” means a volume of a liquid, solution or suspension, intended to be subjected to qualitative or quantitative determination of any of its properties, such as the presence or absence of a component, the concentration of a component, e.g., an analyte, etc. The embodiments of the present invention are applicable to human and animal samples of whole blood. Typical samples in the context of the present invention as described herein include blood, plasma, serum, suspensions thereof, and haematocrit.

The term “about” as used in connection with a numerical value throughout the description and claims denotes an interval of accuracy, familiar and acceptable to a person skilled in the art. The interval governing this term is preferably +10%. Unless specified, the terms described above are not intended to narrow the scope of the invention as described herein and according to the claims.

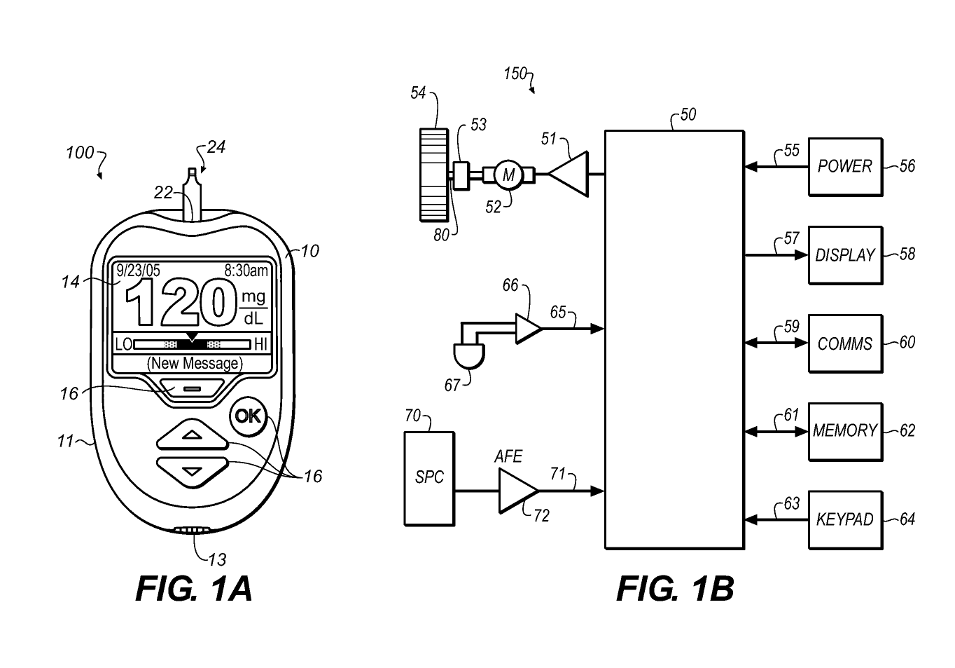

With reference to FIG. 1A there is illustrated an analyte measurement system 100 that includes an analyte or test meter 10 and a test strip 24 that is used with the test meter 10. The analyte meter 10 is defined by a housing 11 that includes a test strip port 22 for receiving one end of the test strip 24. According to one embodiment, the analyte meter 10 may be a blood glucose meter and the test strip 24 is provided in the form of a glucose test strip 24 insertable into the test strip port 22 for performing blood glucose measurements. The analyte meter 10 further includes a plurality of user interface buttons, or keypad, 16 and a display 14, each disposed on a front facing side of the housing 11 as well as a data port 13, as illustrated in FIG. 1A, disposed on a bottom facing side of the housing 11 and opposite the test strip port 22 according to this exemplary embodiment. The positioning of the foregoing features of the test meter 10 can easily be varied. A predetermined number of glucose test strips 24 may be stored in the housing 11 and made accessible for use in blood glucose testing. The plurality of user interface buttons 16 can be configured to allow the entry of data, to prompt an output of data, to navigate menus presented on the display 14, and to execute commands. Output data can include, for example, values representative of an analyte concentration that are presented on the display 14. User inputs may be requested via programmed prompts presented on the display 14, and a user's responses thereto may initiate command execution or may include data that may be stored in a memory module of the analyte meter 10.

Specifically, and according to this exemplary embodiment, the user interface buttons 16 include markings, e.g., up-down arrows, text characters “OK”, etc, which allow a user to navigate through the user interface presented on the display 14. Although the buttons 16 are shown herein as separate switches, a touch screen interface on display 14 with virtual buttons may also be utilized. The display 14 may comprise a movable type of display, such as a sliding display or a tiltable display.

The electronic components of the glucose measurement system 100 can be disposed on, for example, a printed circuit board situated within the housing 11 and forming a data management unit 150 of the herein described system 100. FIG. 1B illustrates, in simplified schematic form, several of the electronic subsystems disposed within the housing 11 for purposes of this embodiment. The data management unit 150 includes a processing unit 50 in the form of a microprocessor, a microcontroller, an application specific integrated circuit (“ASIC”), a mixed signal processor (“MSP”), a field programmable gate array (“FPGA”), or a combination thereof, and is electrically connected to various electronic modules included on, or connected to, the printed circuit board, as will be described below. In one embodiment, the processing unit 50 may comprise a microcontroller such as a model STM32F4 series manufactured by ST Microelectronics of Geneva, Switzerland.

According to this exemplary embodiment, the processing unit 50 is electrically connected to a test strip port connector (“SPC”) circuit 70, that is positioned in the test strip port 22, via an analog front end (AFE) subsystem 72. The analog front end subsystem 72 is electrically connected to the SPC circuit 70 during blood glucose testing. To measure a selected analyte concentration, the SPC circuit 70 detects a resistance or impedance across electrodes of the analyte test strip 24 having a blood sample disposed in a sample chamber 34 therein, using a potentiostat or transimpedance amplifier, and converts an electric current measurement into digital form for presentation on the display 14, typically in units of milligrams per deciliter (mg/dl) or millimoles per liter (mmol/l). The processing unit 50 can be configured to receive input from the SPC circuit 70 via analog front end subsystem 72 over an interface 71 and may also perform a portion of the potentiostat function and the current measurement function.

The analyte test strip 24 can be in the form of a test strip for measuring a glucose concentration, or other analyte appropriate for monitoring of a biological condition, comprising an electrochemical cell, or sample chamber. The test strip 24 is defined by one or more nonporous, non-conducting substrates, or layers, onto which one or more electrodes, or conductive coatings may be deposited. These electrodes may function as working electrodes, reference electrodes, counter electrodes or combined counter/reference electrodes. Additional non-conducting layers may be applied in order to define the planar dimensions of the electrode structure(s). Test strip 24 can also include a plurality of electrical contact pads, where each electrode can be in electrical communication with at least one electrical contact pad. The strip port connector 104 can be configured to electrically interface to the electrical contact pads, using electrical contacts in the form of flexible conductive prongs, and form electrical communication with the electrodes. The test strip 24 can include a reagent layer that is disposed over at least one electrode in the electrochemical cell, including the working electrode. The reagent layer can include an enzyme and a mediator. Exemplary enzymes suitable for use in the reagent layer include glucose oxidase, glucose dehydrogenase (with pyrroloquinoline quinone co-factor, “PQQ”), and glucose dehydrogenase (with flavin adenine dinucleotide co-factor, “FAD”). Enzymes other than those used to determine glucose are also applicable, for example, lactate dehydrogenase for lactate, β-hydroxybutyrate dehydrogenase for β-hydroxybutyrate (ketone body). An exemplary mediator suitable for use in the reagent layer includes ferricyanide, which in this case is in the oxidized form. Other mediators may be equally applicable, depending upon the desired strip operating characteristics, for example, ferrocene, quinone or osmium-based mediators. The reagent layer can be configured to physically transform glucose into an enzymatic by-product and in the process generate an amount of reduced mediator (e.g., ferrocyanide) that is proportional to the glucose concentration. The working electrode can then be used to measure a concentration of the reduced mediator in the form of a current magnitude. In turn, microcontroller 50 can convert the current magnitude into a glucose concentration whose numerical value (in mg/dl or mmol/l) may be presented on the display 14. An exemplary analyte meter performing such current measurements is described in U.S. Patent Application Publication No. US 2009/0301899 A1 entitled “System and Method for Measuring an Analyte in a Sample”, which is incorporated by reference herein as if fully set forth in this application.

Still referring to FIG. 1B, a detector circuit comprising a proximity detector 67 and an amplifier 66 is connected to the processing unit 50 via a signal line 65. The proximity detector 67 is disposed proximate the opening to the test strip port 22 to detect a test strip 24 in proximity to the test strip port 22 opening. As the test strip 24 is inserted into the test strip port 22, the proximity detector 67 senses the presence of the test strip 24 and, in response, transmits an electric signal through the amplifier 66 over the signal line 65 to the processing unit 50. According to at least one version, the proximity detector 67 may include a photo-emitter which emits light of a particular wavelength, for example, a low power LED emitting infra-red light, and a photodetector selected for detecting the wavelength of the emitted light. In the latter version, the inserted test strip 24 interferes with, or breaks, the emitted light from the photo-emitter, which interference is sensed by the photo-detector and results in the proximity detector 67 generating a signal transmitted to the processing unit 50.

The processing unit 50 may be programmed to activate a test strip drive mechanism in response to receiving the signal from the proximity detector 67. In one embodiment, the test meter 10 may remain in a sleep or passive mode until the meter 10 is activated by the signal from the proximity detector 67. In one embodiment, the drive mechanism comprises a motor 52 connected to a motor drive 51, or motor controller, which regulates a direction and speed of the motor 52 via programmed control signals which are transmitted by the processing unit 50. According to the herein described embodiment, a gear box 53, or gear assembly, is attached to the output of the motor 52 for stepping down the rotation drive ratio generated by the motor 52. Attached to the gear box 53 via a drive shaft 80, is a rotatable drive wheel 54, which may comprise a substantially rigid rim covered by a rubber or other suitably compliant layer, or the drive wheel 54 may be comprised mostly of rubber or compliant material sufficient to provide traction when the drive wheel 54, during rotation, physically contacts a portion of the test strip 24 in order to pull the test strip 24 into the test strip port 22 and into engagement with the SPC circuit 70.

A display module 58, which may include a display processor and display buffer, is electrically connected to the processing unit 50 over the communication interface 57 for receiving and displaying output data, and for displaying user interface input options under control of processing unit 50. The display interface is accessible by processing unit 50 for presenting menu options to a user of the blood glucose measurement system 100. User input module 64 may receive responsive inputs from the user manipulating buttons, or keypad 16, which are processed and transmitted to the processing unit 50 over the communication interface 63. The processing unit 50 may have electrical access to a digital time-of-day clock connected to the printed circuit board for recording dates and times of blood glucose measurements and user inputs, which may then be accessed, uploaded, or displayed at a later time as necessary.

An on-board memory module 62, that includes but is not limited to volatile random access memory (“RAM”), a non-volatile memory, which may comprise read only memory (“ROM”) or flash memory, and may be connected to an external portable memory device via a data port 13, is electrically connected to the processing unit 50 over a communication interface 61. External memory devices may include flash memory devices housed in thumb drives, portable hard disk drives, data cards, or any other form of electronic storage device. The on-board memory can include various embedded applications executed by the processing unit 50 for operation of the analyte meter 10, as explained herein. On board or external memory can also be used to store a history of a user's blood glucose measurements including dates and times associated therewith. Using the wireless transmission capability of the analyte meter 10, or the data port 13, as described herein, such measurement data can be transferred via wired or wireless transmission to connected computers or other processing devices.

A communications module 60 may include transceiver circuits for wireless digital data transmission and reception, and is electrically connected to the processing unit 50 over communication interface 59. The wireless transceiver circuits may be in the form of integrated circuit chips, chipsets, and programmable functions operable via processing unit 50 using on-board memory, or a combination thereof. The wireless transceiver circuits may be compatible with different wireless transmission standards. For example, a wireless transceiver circuit may be compatible with the Wireless Local Area Network IEEE 802.11 standard known as WiFi. A transceiver circuit may be configured to detect a WiFi access point in proximity to the analyte meter 10 and to transmit and receive data from such a detected WiFi access point. A wireless transceiver circuit may be compatible with the Bluetooth protocol and is configured to detect and process data transmitted from a Bluetooth “beacon” in proximity to the analyte meter 10. A wireless transceiver circuit may be compatible with the near field communication (“NFC”) standard and is configured to establish radio communication with, for example, an NFC compliant master device in proximity to the analyte meter 10. A wireless transceiver circuit may comprise a circuit for cellular communication with cellular networks and is configured to detect and link to available cellular communication towers.

A power supply module 56 is electrically connected to all modules in the housing 11 and to the processing unit 50 to supply electric power thereto. The power supply module 56 may comprise standard or rechargeable batteries, or an AC power supply that may be activated when the analyte meter 10 is connected to a source of AC power. The power supply module 56 is also electrically connected to processing unit 50 over the communication interface 55 such that processing unit 50 can monitor a power level remaining in a battery of the power supply module 56.

In addition to connecting external storage for use by the analyte meter 10, the data port 13 can be used to accept a suitable connector attached to a connecting lead, thereby allowing the analyte meter 10 to be connected to an external device such as a personal computer. Data port 13 can be any port that allows for transmission of data, power, or a combination thereof, such as a serial, USB, or a parallel port.

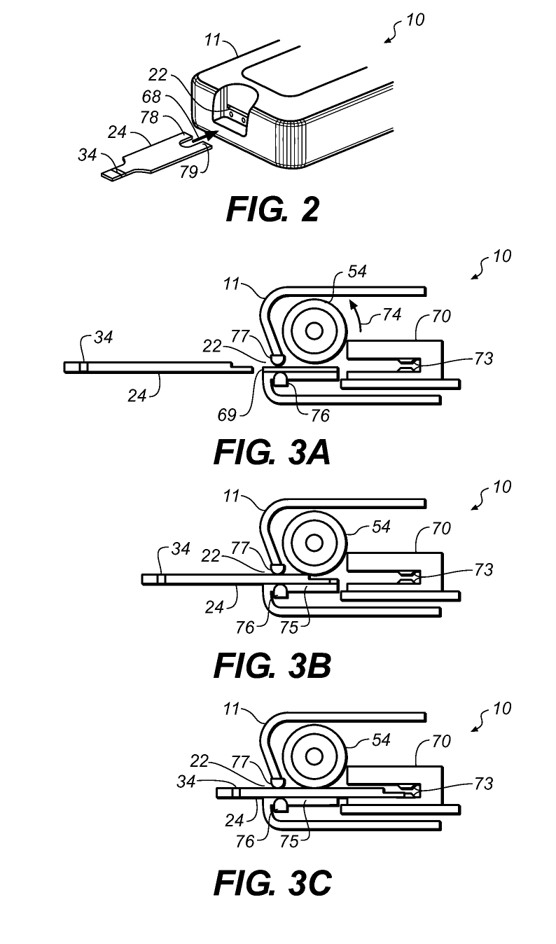

With reference to FIG. 2, there is illustrated a top perspective view of the test meter 10 wherein a test strip 24 is being inserted into the test strip port 22 in the direction indicated by the arrow 68. In this embodiment, the test strip port 22 comprises an opening, in the form of a slot, in one side of the housing 11 of the test meter 10. According to this exemplary embodiment, the test strip 24 comprises electrical contact pads 78, 79, at one end of the test strip 24. The contact pads 78, 79, may be disposed on a top surface of the test strip 24, a bottom surface, or a combination thereof. Proximate to the test strip port 22, there is disposed the proximity detector 67 comprising the photo-emitter 77 and photo-detector 76 (FIG. 3A). The proximity detector 67 detects the presence of the test strip 24, in which the end having the contact pads 78, 79, is inserted into the test strip port 22. Upon activation by the presence of the test strip 24, the proximity detector 67 transmits a signal to the processing unit 50 which activates the motor 52 to cause the drive wheel 53 to rotate in a first direction in order to pull the test strip 24 into the test meter 10 and an assay position with the SPC circuit 70. The rotation of the drive wheel 53 stops when the test strip 24 reaches the assay position, i.e., when the electrical contact pads 78, 79 of the test strip 24 make an electrical connection with the electrical contacts 73 (FIG. 3A) of the SPC circuit 70. A portion of the test strip 24, including the sample chamber 34, or electrochemical cell, remains accessible outside of the test meter housing 11 at an opposing end of the test strip 24 as shown in FIG. 2 so that the sample chamber 34 may receive a sample provided by the user of the test meter 10. After the user applies a sample to an inlet of the sample chamber 34, the sample is detected by the SPC circuit 70 and an assay sequence is initiated by the processing unit 50 as in the usual course.

With reference to FIGS. 3A-3C, sequential views of the operation of the herein described drive mechanism are provided in which a test strip 24 is first brought into proximity to the test strip port 22 of the test meter 10 (FIG. 3A). As the test strip 24 nears the test strip port 22, the photo-detector/photo-emitter pair 76, 77, detects the presence of the edge of the test strip 24 which blocks the photo-emitter light beam whereby a detection signal is then transmitted to the processing unit 50. In response to the detection signal, the processing unit 50 sends its own activation signal to the motor drive interface 51 which initiates rotation of the drive wheel 54 in the direction shown by the arrow 74. The drive wheel 54 is positioned proximate an interior planar surface 75 disposed within the confines of the test strip port 22 such that the test strip 24 is pinched between the interior surface 75 and the rotating drive wheel 54 (FIG. 3B). This pinching action against the test strip 24 provided by the compliant exterior surface of the drive wheel 54 against surface 75 creates a gripping pressure upon the surface of the test strip 24 whereby the test strip 24 is pulled inwardly and in registration with a guide rail 69 and into electrical engagement with the SPC circuit electrical contacts 73. The SPC circuit 70 is positioned at a distance within the test meter housing 11 such that when the test strip 24 is pulled into the SPC circuit 70 and the test strip contact pads 78, 79, make an electrical connection with the electrical contacts 73 therein, a portion of the test strip 24 remains outside the test meter housing 11 so that access can be had to the sample chamber 34 by a user of the test meter 10. After the user applies a sample to the sample chamber 34, the processing unit 50 may receive a sample detection signal indicating the presence of the sample and, in response to the sample detection, the processing unit 50 may trigger a sequence of programmed steps for performing an assay on the provided sample. In one embodiment and upon completion of the sample assay, the processing unit 50 may transmit a signal to the motor drive 51 to reverse rotation of the drive wheel 54 for ejecting the test strip 24 from the test strip port 22.

In one embodiment, a drive wheel 54 comprising a smooth, compliant surface for making contact with the test strip 24, and having a width greater than the width of the test strip, such that the sides of the drive wheel 54 extend beyond both side edges of the test strip 24, could be used to form a sample fluid barrier, or sealing feature, to prevent the sample applied to the test strip from leaking into the strip port connector circuit 70. The test strip 24 would fit snugly between a pair of guide rails 69 at the lateral sides of the strip, reducing available channels for fluid contamination to flow past the drive wheel 54 towards the strip port connector circuit 70. The height of the drive wheel 54 above the test strip 24 and the height of the guide rails 69 may be selected so that the drive wheel 54 makes contact with the upper surface of the test strip 24 when inserted, while not rubbing against the guide rails. The pressure from the drive wheel 54 against the test strip 24 in conjunction with the guide rails 69 serves to confine any leaked sample fluid away from the strip port connector circuit 70. In addition, the test strip 24 itself may be modified to facilitate traction against the drive wheel 54. Although the engaged test strip 24 may be essentially planar, a top layer of the test strip 24, i.e. the surface upon which the drive wheel makes contact, may further include at least one feature such as nubs, or cross-wise grooves, or other protruding or recessed physical features that could cooperate with the engaging drive wheel 54 to aid in retraction and/or ejection of the test strip 24.

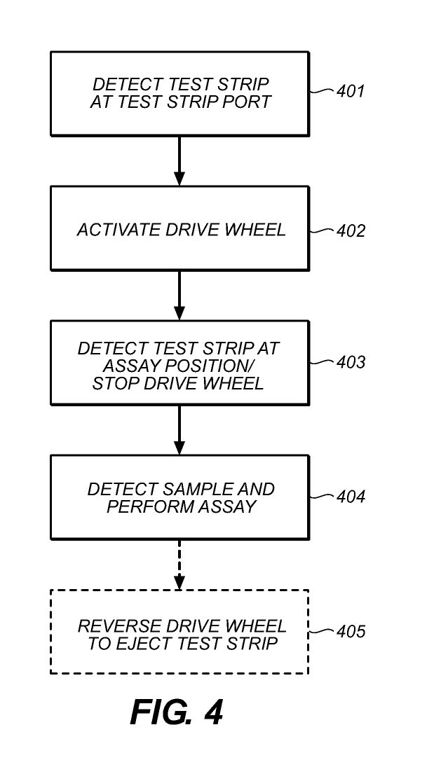

With reference to FIG. 4, there is illustrated a flowchart illustrating exemplary steps performed by the test meter 10 in connection with a test strip 24. At step 401, the presence of a test strip 24 being inserted into the test strip port 22 of the test meter 10 is initially detected by the proximity detector 67. In response to this detection, the proximity detector 67 generates a signal transmitted to the processing unit 50 which, in turn, activates the drive wheel 54, at step 402. More specifically, the processing unit 50 transmits a signal to the motor control circuit 51, which causes the motor 52 to rotate the drive wheel 54 in a first (intake) direction, contacting the inserted test strip 24 and drawing the test strip 24 inwardly toward and into engagement with the SPC circuit 70 and into an assay position in which the test strip 24 is suitably connected to the electrical contacts 73 therein. The electrical connection between the test strip 24 and the electrical contacts 73 is detected by the processing unit 50, at step 403, which transmits a signal to the motor drive interface 51 to immediately stop the rotation of the drive wheel 54. After the test strip 24 reaches the assay position (FIG. 3C), the test meter 10 awaits application of a sample to the sample chamber 34. Upon detecting application of a sample, the test meter 10 initiates an assay sequence for measuring an analyte concentration of the provided sample. At alternative step 405, after an analyte measurement is completed—typically within five seconds, the test meter 10 may be programmed to send an eject signal to the motor drive interface 51 which causes the motor 52 to reverse its rotation and to eject the test strip 24.

As will be appreciated by one skilled in the art, aspects of the present invention may be embodied as a processing system, method, or apparatus. Accordingly, aspects of the present invention may take the form of an entirely hardware embodiment, an entirely software embodiment (including firmware, resident software, micro-code, etc.), or an embodiment combining software and hardware aspects that may all generally be referred to herein as a “circuit,” “circuitry,” “module,” “subsystem” and/or “system.” Furthermore, aspects of the present invention may take the form of a computer program product embodied in one or more computer readable medium(s) having computer readable program code embodied thereon.

Program code and/or data representative of operations and measurements performed may be stored using any appropriate medium, including but not limited to any combination of one or more computer readable medium(s). A computer readable storage medium may be, for example, an electronic, magnetic, optical, electromagnetic, infrared, or semiconductor system, apparatus, or device, or any suitable combination of the foregoing. More specific examples of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible, non-transitory medium that can contain, or store a program for use by or in connection with an instruction execution system, apparatus, or device.

Program code and/or data representative of operations and measurements performed may be transmitted using any appropriate medium, including but not limited to wireless, wireline, optical fiber cable, RF, etc., or any suitable combination of the foregoing.

### PARTS LIST FOR FIGS. 1A-4

10 analyte meter
11 housing, meter
13 data port
14 display
16 user interface buttons
22 test strip port
24 test strip
34 sample chamber
50 microcontroller (processing unit)
51 motor drive interface
52 motor, electric
53 gear assembly
54 drive wheel
55 power supply interface
56 power supply module
57 display module interface
58 display module
59 communications interface
60 communications module
61 memory module interface
62 memory module
63 user input interface
64 user input module/keypad
65 signal line
66 amplifier
67 proximity detector
68 arrow
69 guide rail
70 strip port connector circuit
71 strip port connector interface
72 test strip analyte module—analog front end subsystem
73 strip port connector electrical contacts
74 arrow
75 interior surface, test strip port
76 photo-detector
77 photo-emitter
78 contact pad
79 contact pad
80 drive shaft
100 analyte measurement system
150 data management unit
401 step—detect test strip
402 step—activate drive wheel
403 step—detect test strip at assay position/stop drive wheel
404 step—detect sample and perform assay
405 step—reverse drive wheel

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. A test meter comprising:
a test strip port configured for receiving an analytical test strip; and
a drive mechanism for automatically pulling a partially inserted test strip fully into the test strip port and into an operative position in which the test strip is mechanically and electrically connected to the test meter.

2. The test meter of claim 1, wherein the drive mechanism comprises a rotatable drive wheel.

3. The test meter of claim 2, wherein the drive mechanism further comprises an electric motor connected to the drive wheel which is configured to rotate the drive wheel.

4. The test meter of claim 3, further comprising a detector electrically connected to the motor, the detector being disposed relative to the strip port for detecting a presence of a test strip proximate the test strip port and sending a signal to activate the motor in response to detecting the presence of the test strip.

5. The test meter of claim 4, wherein the detector comprises a photodetecting device.

6. The test meter of claim 4, wherein the drive mechanism further comprises a motor control circuit connected to the electric motor for generating a signal to stop the motor's rotation in response to the electrical contact engaging the contact pad.

7. The test meter of claim 6, wherein the motor control circuit comprises a circuit for signaling the motor to rotate the drive wheel in an opposite direction for ejecting the test strip.

8. The test meter of claim 4, wherein the test meter remains in a sleep mode until the detector for detecting the presence of the test strip sends the signal to activate the motor.

9. The test meter of claim 1, wherein the test strip port further comprises a guide rail for aligning with contact pads of the test strip and with electrical contacts provided in the test strip port for creating electrical engagement.

10. A test meter comprising:
a test strip port for receiving an analyte test strip comprising an electrochemical cell;
a detector at an inlet of the test strip port for detecting the test strip; and
a drive mechanism at the inlet of the test strip port connected to the detector for pulling the test strip into the test strip port in response to the detector detecting the presence of the test strip.

11. The test meter of claim 10, wherein the detector comprises a photo-emitter and a photo-detector.

12. The test meter of claim 11, wherein the test strip comprises contact pads electrically connected to the electrochemical cell, the test strip port comprises electrical contacts, and the mechanism comprises a motor control circuit for stopping the test strip when the electrical contacts engage the contact pads.

13. The test meter of claim 10, wherein the drive mechanism comprises a motorized drive wheel controlled by the motor control circuit to rotate in a forward direction and in a reverse direction.

14. The test meter of claim 10, wherein the drive wheel comprises a compliant surface to contact the test strip for pulling the test strip into the test strip port.

15. The test meter of claim 12, wherein the electrochemical cell is accessible for a user to provide a sample therein when the test strip is stopped in the test strip port.

16. A method of operating an analyte test meter, the method comprising:
detecting a presence of a test strip proximate a test strip port of the analyte meter;
activating a drive mechanism in response to the step of detecting for pulling the test strip into the test strip port, the drive mechanism including a drive wheel that engages a portion of the test strip; and
deactivating the drive mechanism when contact pads on the test strip electrically engage contacts in the test strip port.

17. The method of claim 16, further comprising guiding the test strip into the test strip port using a guide rail for aligning an edge of the test strip.

18. The method of claim 17, further comprising determining that a sample is applied to the test strip using the electrical contacts.

19. The method of claim 18, further comprising transmitting an electrical signal through the electrical contacts in the test strip port for performing an assay.

20. The method of claim 19, further comprising activating the drive mechanism in a reverse direction for ejecting the test strip.

21. A hand-held test meter for use with an analytical test strip in the determination of an analyte in a bodily fluid sample, the hand-held test meter comprising:
a strip port connector configured to receive an analytical test strip, the strip port connector including:
a test strip proximity sensor; and
a test strip drive mechanism,
wherein the test strip proximity sensor is configured to sense an analytical test strip approaching the strip port connector and to activate the test strip drive mechanism, and
wherein the test strip drive mechanism is configured to automatically draw the approaching analytical test strip into the strip port connector once activated by the test strip proximity sensor.
